(19) United States
(12) Patent Application Publication
(10) Pub. No.: US 2003/0153926 A1
(43) Pub. Date: Aug. 14, 2003

(54) Distracting cannula for sheathless arthroscope

(76) Inventors: Reinhold Schmieding, Naples, FL (US); James P. Bradley, Pittsburgh, PA (US)

Correspondence Address: DICKSTEIN SHAPIRO MORIN & OSHINSKY LLP, 2101 L STREET NW, WASHINGTON, DC, 20037-1526, US

(21) Appl. No.: 10/359,650

(22) Filed: Feb. 7, 2003

Related U.S. Application Data
(60) Provisional application No. 60/354,500, filed on Feb. 8, 2002.

(51) Int. Cl. A61F 11/00

(52) U.S. Cl. 606/108

(57) ABSTRACT

An arthroscope cannula for a sleeveless arthroscope. The arthroscope cannula is provided with an elongated shaft, a distal end with a threaded portion which allows the arthroscope cannula to securely lock into the knee capsule, and a proximal end for receiving the sheathless arthroscope. The cannula includes a slidable retention clip that is advanced on the shaft of the cannula to a position against the skin to maintain traction and stabilize the cannula against retraction, and to distend the joint capsule.

This application claims the benefit of U.S. Provisional Application Serial No. 60/354,500, filed Feb. 8, 2002, the entire disclosure of which is incorporated herein by reference.

### FIELD OF THE INVENTION

The present invention relates to surgical devices and procedures and, more specifically, to a sheathless arthroscope and distracting cannula for use during arthroscopic surgery and method of conducting arthroscopic surgery.

### BACKGROUND OF THE INVENTION

In arthroscopic surgery, arthroscopes are employed to visually inspect a joint, such as a knee or shoulder joint, and to conduct diagnostic viewing of tissue or cartilage within the joint. Arthroscopes typically include a lens system for visualizing the affected area, as well as a fiber optic passage for transmitting light for illuminating the desired area.

In the conventional method of using an arthroscope, the arthroscope is inserted in a sheath or sleeve (for protection and rigidity) and is inserted directly into the joint through an incision. Movement of the arthroscope during surgery can cause skin irritation and, more importantly, the additional width of the instrument provided by sheath limits the use of the arthroscope in small joints and limits accessibility to small structures within the joint.

Accordingly, there is a need for a system which allows an arthroscope to be inserted into a joint without a protective sheath.

### SUMMARY OF THE INVENTION

In accordance with the present invention, a cannula is provided which is specifically designed to allow an arthroscope to be used without a sheath. The inner end of the cannula tube is provided with one complete thread which is rotated through the capsule of the joint (such as a knee joint) to secure the cannula against the inner surface of the capsule. The cannula is further provided with a slidable retention member, preferably in the form of a clip, which is pushed up against the skin to maintain traction and stabilize the cannula. As the clip is pushed forwardly, the capsule is distracted, providing a larger working space within the joint.

The cannula and sheathless arthroscope of the present invention provide the following advantages:

1. The cannula provides a channel through the tissue for insertion of the sheathless arthroscope, eliminating irritation of the skin as the arthroscope is manipulated in the patient.

2. The distal thread of the cannula locks the cannula to the capsule, allowing capsule to be distracted to create more working space in the joint.

3. The larger inner diameter of the cannula allows greater inflow of irrigation as compared to the sheath of the arthroscope, providing better vision and distention control.

4. An arthroscope with a stronger (thicker) side wall is required, but this can be accomplished while maintaining the standard 4 mm diameter by using an arthroscope with less fiber bundles.

5. The elimination of the arthroscope sheath allows the surgeon to access smaller joints and to better position the scope in the joint to visualize structures not ordinarily accessible when using a standard sized sheath.

These and other features and advantages of the invention will become apparent from the following detailed description that is provided in connection with the accompanying drawings and illustrated exemplary embodiments of the invention.

### BRIEF DESCRIPTION OF THE DRAWINGS

FIG. 1 illustrates a schematic top view of an arthroscope cannula of the present invention.

FIG. 2 illustrates a cross-sectional view of the arthroscope cannula of FIG. 1 rotated 90 degrees.

FIG. 3 illustrates a cross-sectional view of the arthroscope cannula of FIG. 1 rotated 180 degrees.

FIG. 4 illustrates a perspective view of a clip to be attached to the arthroscope cannula of FIG. 1 in accordance with the present invention.

FIG. 5 illustrates top view of the clip of FIG. 4.

FIG. 6 illustrates a perspective view of a sheathless arthroscope inserted in the cannula of the present invention.

FIG. 7 illustrates a perspective view of the sheathless arthroscope, cannula inserted through an incision in skin, with the clip advanced to distract the capsule.

FIG. 8 illustrates a perspective view of the cannula of the present invention with an obturator inserted through the elongated tube.

FIG. 9 illustrates a cross-sectional view of the cannula and obturator assembly of FIG. 8.

FIG. 10 illustrates a view of the cannula and obturator assembly of FIG. 9 rotated 90 degrees.

### DETAILED DESCRIPTION OF THE PREFERRED EMBODIMENT

In the following detailed description, reference is made to various specific embodiments in which the invention may be practiced. These embodiments are described with sufficient detail to enable those skilled in the art to practice the invention, and it is to be understood that other embodiments may be employed, and that structural and logical changes may be made without departing from the spirit or scope of the present invention.

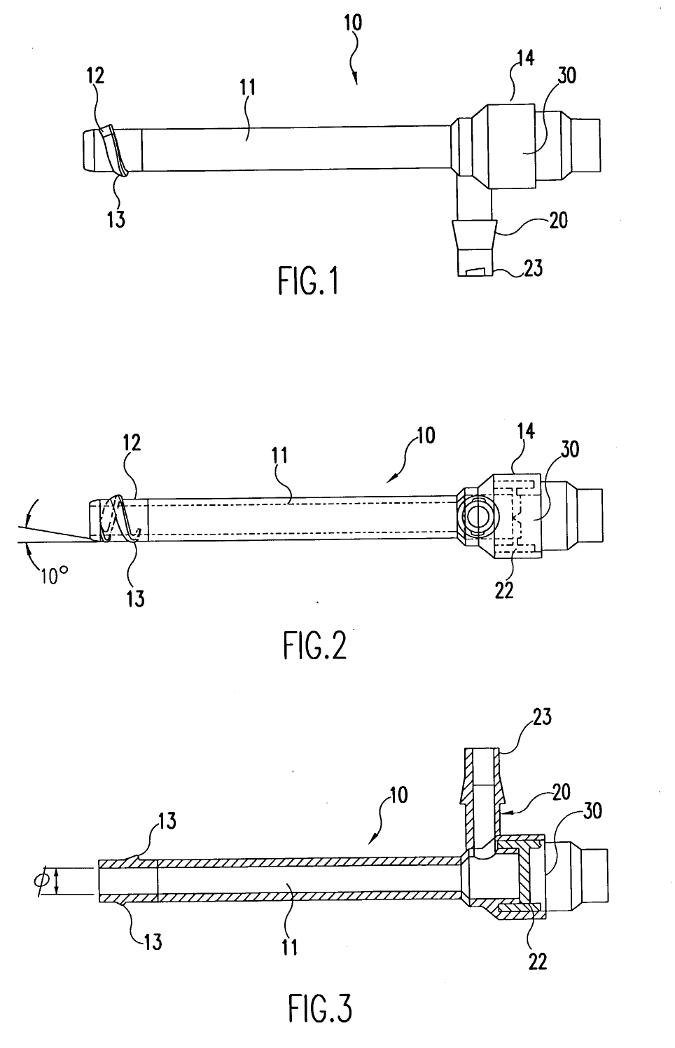

Referring now to the drawings, where like elements are designated by like reference numerals, FIGS. 1-3 illustrate a cannula 10 of the present invention for receiving an arthroscope, which allows the arthroscope to be inserted in the and securely positioned within a knee joint, for example, without an outer tubular sheath or sleeve.

As described in more detail below, the arthroscope cannula is provided at its distal end with a threaded tip that is screwed through the capsule of the joint. After the threaded tip of the cannula is secured within the capsule of the joint, a slidable retention clip on the cannula is axially advanced toward the distal end of the cannula and pushed up against the patient's skin, to stabalize the cannula and distract the capsule.

The arthroscope cannula 10 preferably comprises an elongate, narrow diameter body or shaft 11. Preferably, the arthroscope cannula 10 of the present invention is employed in arthroscopic surgeries such as knee, shoulder or elbow arthroscopies. The arthroscope cannula preferably has a length of about 7 cm. Elongated shaft is tubular and preferably provided in two versions with different inner diameters “ø”, either 5.75 millimeters or or 8.25 millimeters. In both versions, the inner diameter is slightly tapered along its length.

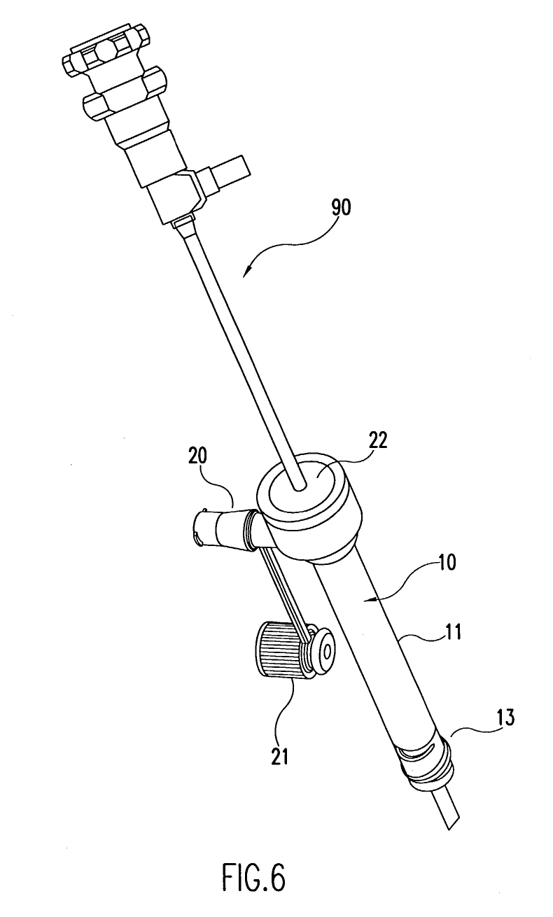

As illustrated in FIGS. 1-3, the arthroscope cannula 10 has a proximal end 14 and a distal end 12, which is provided with one complete thread 13. A fluid passage 20 is located toward the proximal end 14 and extends from the elongated shaft 11, as shown in FIGS. 1 and 3. The fluid passage 20 is tubular and is connected to a source of irrigation solution for supplying irrigation through the cannula to the work site during the arthroscopic procedure. If desired, a sealing means, such as a plastic cap 21 (FIG. 6), for example, may be optionally employed to securely engage distal end 23 of the passage 20.

Referring to FIGS. 1-3 and 9, a transluscent silicone dam 22 and a coupling member 29 are disposed within an enlarged opening 30 (FIGS. 1-3) located at the proximal end 14 of the cannula. The dam 22 serves to seal the cannula passage to prevent fluid leakage around an arthroscope or other instrument. Coupling member 29 may be provided with a threaded portion 31 for engaging corresponding threads on an obturator, as described below.

Preferably, cannula 10 and coupling member 29 are formed of a durable, transluscent polycarbonate material.

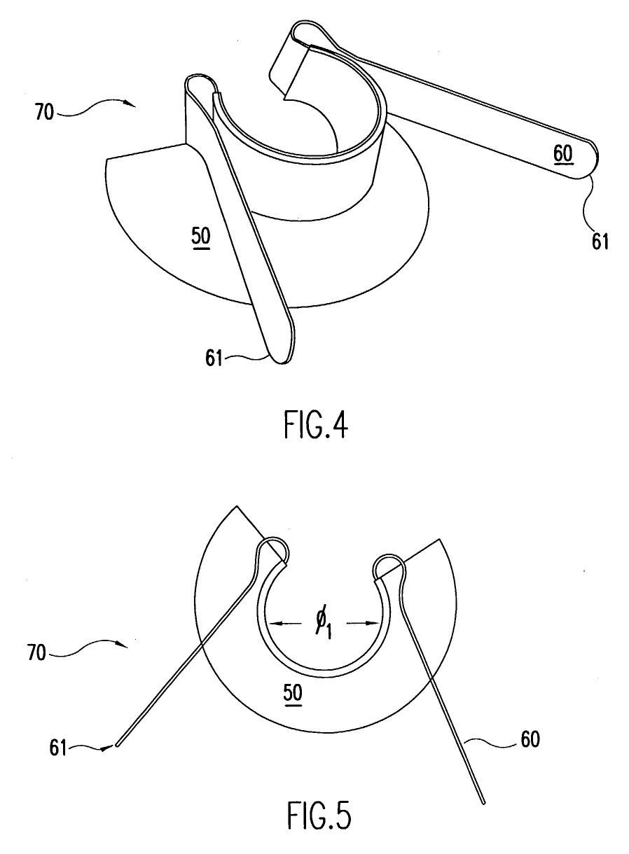

Arthroscope cannula 10 of the present invention is employed in conjunction with a slidable retention member 70 (FIGS. 4-5) in the form of a clip, comprising a clear PVC retention collar 50 and a flexible stainless steel spring member 60. The inner diameter “ø₁” of the retention clip 70 is about equal to the outer diameter of the elongated shaft 11 of the arthroscope cannula 10 so that the clip can be slidably mounted on the shaft.

To mount and axially move the clip along the shaft of the arthroscopic cannula, the distal ends 61 of the flexible wire member 60 are squeezed together so that retention collar 50 flexes open. In its flexed position, the clip is then able to be inserted over the elongated shaft 11 of arthroscope cannula 10 and slid up snugly against the patient's skin, after which the ends 61 are released to close the clip around the shaft and secure the clip in place.

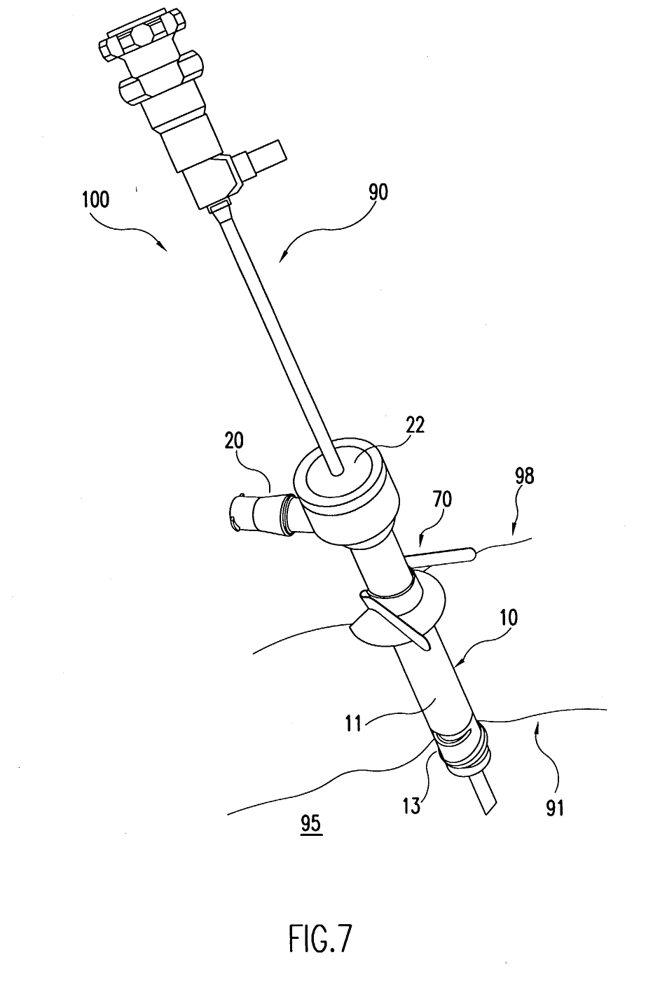

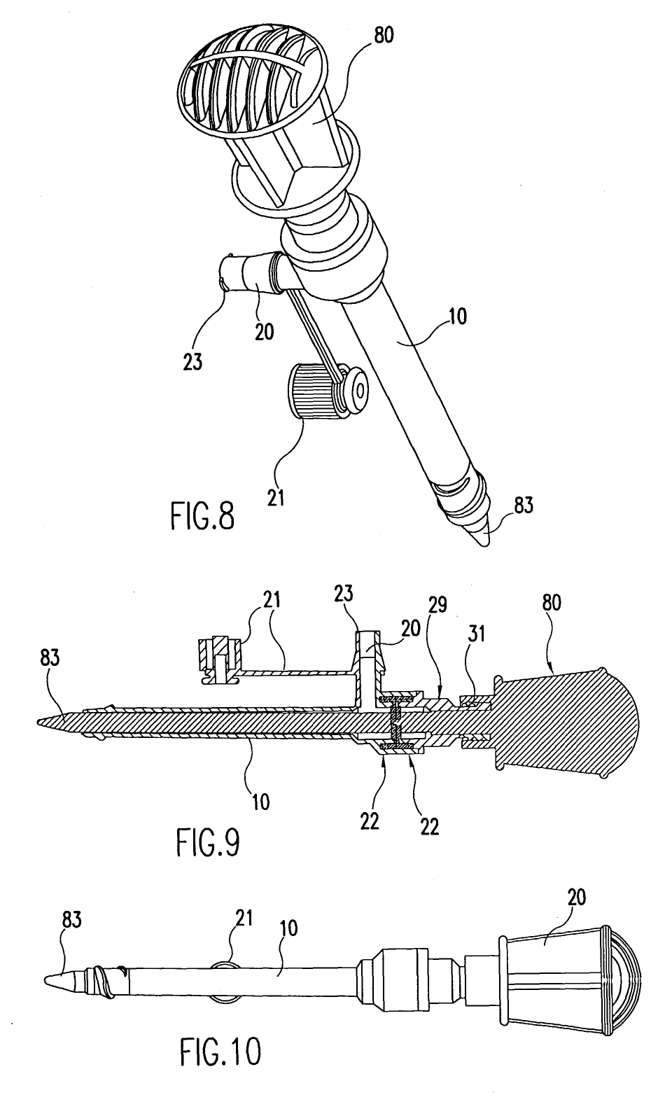

The arthroscope cannula 10 of the present invention may be employed in a variety of endoscopic procedures, preferably, in arthroscopic interventions such as knee, joint or elbow arthroscopies. To better illustrate an exemplary surgical procedure conducted with the arthroscope cannula 10 of the present invention, reference is made to FIG. 7, which illustrates the tip of the cannula inserted through and distracting a capsule of a knee joint 35. An incision is first made in the knee 95 of interest. An obturator, such as obturator 80 of FIGS. 8-10, may be inserted into the stabilized arthroscope cannula 10 to facilitate insertion of the cannula through the incision. The obturator 80 is provided at its distal end with a blunt tip or nose 83 which guides the cannula 10 through the surgical incision. The arthroscope cannula 10 is gently threaded through the incision into knee capsule 91 to allow the threaded portion 13 of the elongated shaft 11 to be securely positioned behind the knee capsule 91.

Once the cannula is inserted into the desired knee region, obturator 80 is removed from the cannula 10, and retention clip 70 is mounted onto the shaft of the cannula. The retention clip 70 is axially advanced toward the distal end 12 of the cannula 10 and pushed up against patient's skin 98. As noted above, the retention assembly maintains traction and confers stability to the cannula, while simultaneously distracting the joint capsule to provide a larger working space.

After the cannula is secured in place, a sleeveless arthroscope 90, preferably a 4 mm arthroscope, is inserted in the cannula 10 to form the arthroscopic cannula/scope assembly 100 illustrated in FIG. 7. The arthroscope 90, connected to a light source, is appropriately positioned within the joint to provide pictorial display of the surgical site on a television screen (not shown) visible to the surgeon.

The above description and drawings illustrate preferred embodiments which achieve the objects, features and advantages of the present invention. It is not intended that the present invention be limited to the illustrated embodiments. Any modification of the present invention which comes within the spirit and scope of the following claims should be considered part of the present invention.

What is claimed as new and desired to be protected by Letters Patent of the United States is:

1. An arthroscope cannula, comprising:
an elongated cannulated shaft having a proximal end and a distal end;
a threaded region located at the distal end of the elongated shaft; and
a slidable retention member mounted on the cannulated shaft.

2. The arthroscope cannula of claim 1, wherein the retention member comprises a retention ring and a flexible wire.

3. The arthroscope cannula of claim 1, wherein the elongated shaft is formed of a translucent material.

4. The arthroscope cannula of claim 1, wherein the elongated shaft has an inner diameter of about 5.75 millimeters.

5. The arthroscope cannula of claim 1, wherein the elongated shaft has an inner diameter of about 8.25 millimeters.

6. The arthroscope cannula of claim 1, wherein the elongated shaft accommodates a sleeveless 4 millimeter arthroscope.

7. The arthroscope cannula of claim 1, further comprising a fluid passage provided at the proximal end of the elongated shaft.

8. An arthroscopic system, comprising:
an elongated cannulated shaft having a proximal end and a distal end;
a threaded region located at the distal end of the elongated shaft; and
a slidable retention member attached to the cannulated shaft; and
a 4 millimeter arthroscope insertable within the cannulated shaft of the cannula.

9. The arthroscopic system of claim 8, wherein the cannula is formed of a translucent polymer.

10. The arthroscopic system of claim 8, further comprising an obturator insertable in the cannulated shaft.

11. A method of conducting arthroscopic surgery, comprising the steps of:
providing an incision in tissue at a joint location;
inserting an arthroscope cannula through the incision, the arthroscope cannula being provided with an elongated shaft having a proximal end and a distal end; a threaded region located at the distal end; and a slidable retention member;
securing the threaded region of the arthroscope cannula into a joint capsule;
sliding the retention member toward the distal end of the cannula to stabilize the cannula and cause distention of the capsule; and
inserting an arthroscope into the cannula and through the incision to observe a surgical procedure within the joint capsule.

12. The method of claim 11, further comprising the steps of:
inserting an obturator into the cannula prior to inserting the cannula in the incision, and
removing the obturator prior to inserting the arthroscope into the cannula.
